(19) United States
(12) Patent Application Publication
(10) Pub. No.: US 2022/0125872 A1
(43) Pub. Date: Apr. 28, 2022

(54) POLYHERBAL TRANSDERMAL PATCH FOR PAIN MANAGEMENT AND ITS PROCESS OF PREPARATION

(71) Applicants: Srinivas Reddy MALE, Hyderabad (IN); Praveen Kumar MANDAPALLI, Hyderabad (IN); V S Vinai Kumar TENNETI, Hyderabad (IN); Shantaram Laxman PAWAR, Hyderabad (IN); Azista Industries Pvt Ltd, Hyderabad (IN)

(72) Inventors: Srinivas Reddy Male, Hyderabad (IN); Praveen Kumar Mandapalli, Hyderabad (IN); V S Vinai Kumar Tenneti, Hyderabad (IN); Shantaram Laxman Pawar, Hyderabad (IN)

(21) Appl. No.: 17/603,251

(22) PCT Filed: Apr. 11, 2020
(86) PCT No.: PCT/IB2020/053460

§ 371 Date: Oct. 12, 2021

(30) Foreign Application Priority Data
Apr. 13, 2019 (IN) 201941014934

(51) Int. Cl. A61K 36/9068; A61K 36/324; A61K 36/185; A61K 36/484; A61K 9/70

(52) U.S. Cl. CPC A61K 36/9068; A61K 36/324; A61K 9/7053; A61K 36/484; A61K 36/185

(57) ABSTRACT

The present invention relates to composition of polyherbal transdermal patch comprising combination of natural herbal extracts as active ingredients and pharmaceutically acceptable excipients. The present invention relates to composition of polyherbal transdermal patch comprising combination of natural herbal extracts includes boswellia extract, evening primrose oil, blackcurrant seed oil, Ginger, Licorice, Cats claw and Devil's claw and other pharmaceutically acceptable excipients. The present invention also relates to composition of polyherbal transdermal patch comprising boswellia extract, evening primrose oil, blackcurrant seed oil, Ginger, Licorice, Cats claw and Devil's claw as natural herbal ingredients and pharmaceutically acceptable excipients for the treatment/management of pain. The present invention also relates to an efficient process for the preparation of polyherbal transdermal patch by using hot-melt coating technique (HMC), comprising the steps of melting, mixing, coating, laminating, cutting, pouching and labelling.

### FIELD OF THE INVENTION

The present invention relates to composition of polyherbal transdermal patch comprising combination of natural herbal extracts as active ingredients and pharmaceutically acceptable excipients.

The present invention relates to composition of polyherbal transdermal patch comprising combination of natural herbal extracts which includes boswellia extract, evening primrose oil, blackcurrant seed oil, Ginger, Licorice, Cat's claw and Devil's claw and other pharmaceutically acceptable excipients.

The present invention also relates to composition of polyherbal transdermal patch comprising boswellia extract, evening primrose oil, blackcurrant seed oil, Ginger, Licorice, Cat's claw and Devil's claw as natural herbal ingredients and pharmaceutically acceptable excipients for the treatment/management of pain.

The present invention also relates to an efficient process for the preparation of polyherbal transdermal patch by using hot-melt coating technique (HMC), comprising the steps of melting, mixing, coating, laminating, cutting, pouching and labelling.

### BACKGROUND OF THE INVENTION

Formulations in combination of two or more natural herbal extracts as polyherbal formulation is expected to improve overall therapeutic effect by synergism.

Polyherbal formulations have gained lot of attention in traditional Ayurvedic treatment. The dose of each herbal extract can be reduced when the natural extracts were used in combination, which results better therapeutic effect and lesser side effects and the dosage frequency can also be reduced with better patient compliance.

Many natural herbal extracts were used in combination of two or more herbals to provide better therapeutic effect and below given are few references: Tinospora cordifolia, Pterocarpus marsupium, Gymnema sylvestre, Zingiber officinale and Momordica charantia as ayurvedic polyherbal combination for the treatment of diabetes, Zingiber officinale, Piper nigrum, long pepper given as combination for mucous reducing effect, Guduchi and curcumin can be used in combination to improve immunity, Cuminum cyminum, Piper nigrum, asafoetida combination is used to relieve bloating problems.

Arthritis is a long-term autoimmune disorder that primarily affects joints. A common feature of rheumatic diseases is the involvement of joints and the surrounding tissues such as ligaments, tendons and muscles which results swollen and painful joints. Pain and stiffness often worsen following rest and most commonly, the wrist and hands are involved, with the same joints typically involved on both sides of the body.

The inventors of the present invention have developed a transdermal patch using polyherbal extracts including boswellia serrata, evening primrose oil, blackcurrant seed oil, licorice, ginger, cat's claw and devil's claw in combination to provide better effect for the treatment of arthritis. The goal of transdermal route is to maximize the flux through the skin into the systemic circulation and simultaneously minimize the metabolism of the drug. Transdermal route can minimize the first pass metabolism which is associated with oral administration and it has also provides good systemic circulation. Individually each ingredient has proven to be effective in treating pain; hence the effective treatment or management of pain can be expected in combination of all these herbal ingredients.

Boswellia serrata

Boswellia serrata is a medium to large branching tree, generally found in dry hilly areas of India, North Africa and the Middle East, belongs to the family Burseraceae. Boswellia serrata is called Indian olibanum, Indian frankincense or “Dhup”, it is also known as Salai guggul, and Sallaki in Sanskrit.

Boswellia serrata, in Sanskrit is known as Gajabhakshya, implying its ingestion by elephants. Charaka, Bhavamisra and others have described it as useful. It possesses anti-inflammatory, anti-arthritic and analgesic activity.

Boswellia serrata plant contains boswellic acid as its major active constituent which is present as α-boswellic acid; β-boswellic acid; 3-acetyl-11-keto β-boswellic acid (AKBA), responsible for anti-arthritic activity. Boswellia serrata is being used in the management of rheumatoid arthritis, osteoarthritis solely because of these potent active constituents from ancient times. Boswellic acid shows its activity by inhibiting the synthesis of pro-inflammatory cytokines and 5-lipoxygenase activity.

The resin extracted from plant has many pharmacological uses. The oleo-gum resin of Boswellia Serrata is a complex mixture of lower and higher Terpenoids and carbohydrates. Higher Terpenoids, collectively called the Boswellic acids are the major fraction of the resin (25-35%), Boswellic acid 65%-85%.

### Evening Primrose Oil

Evening primrose oil is extracted from the seeds of Oenothera biennis biennial plant belongs to Onagraceae family, is a native to North American and it is effective in inflammatory related diseases, especially arthritis. Evening primrose oil is an excellent source of polyunsaturated omega-6 fatty acids that is made up of gamma-linolenic acid (GLA) and linolenic acid (LA) and smaller amount of oleic acid, palmitic acid stearic acid.

Evening primrose oil contains 2-15% gamma-linolenic acid (GLA) and 70% linolenic acid (LA, which turned into GLA in body), which are types of polyunsaturated omega-6 essential fatty acids. GLA is important for maintaining a joint's cell structure and function, apart from reducing the activation of immune cells-lymphocytes and inhibiting the production of inflammatory agents, it also scavenges the free radicals and inhibits the inflammatory enzyme COX-2.

### Blackcurrant Seed Oil

Blackcurrant seeds also known as quinsy berries, squinsy berries, red currant, mustaherukka, grosellero negro, siyah frenkuzumu.

Blackcurrant seed oil is extracted from seeds of Ribes nigrum is a woody shrub in the family of Saxifragaceae, grown for its berries. It is native to temperate parts of central and northern Europe and northern Asia. Oil produced from blackcurrant seeds contains 15-20% gamma-linolenic acid (GLA). GLA is an essential fatty acid that's important for maintaining a joint's cell structure and function. Body converts it into hormone-like substances called prostaglandins, which regulate your immune system and fight joint inflammation. GLA might also suppress inflammatory responses by directly acting on some inflammatory cells.

### Licorice

Licorice is the root of Glycyrrhiza glabra plant which belongs to the family Fabaceae, is a herbaceous perennial legume native to the Middle East, southern Europe, and parts of Asia, such as India, and the plant contains glycyrrhizic acid, or GZA. GZA is made of one molecule of glycyrrhetinic acid and two molecules of glucuronic acid.

Glycyrrhizic acid or glycyrrhizin (GL), is a natural and major pentacyclic triterpenoid glycoside, major bioactive components of licorice on the biological effects, particularly their anti-inflammatory and anti-arthritic effects. It is well known that cyclooxygenase (COX)-2 is an important target of licorice, as many constituents of licorice are able to suppress COX-2, which is critically involved in the inflammatory diseases like RA.

### Ginger

Ginger (Zingiber officinale) is proven effective in decreasing inflammation, swelling, and pain. Gingerols are the major constituents of fresh ginger and are found slightly reduced in dry ginger, whereas the concentrations of shogaols, which are the major gingerol dehydration products, are more abundant in dry ginger than in fresh ginger.

Ginger is known to suppress prostaglandin (a pro-inflammatory molecule) synthesis by inhibition of the enzyme cyclooxygenase (COX-1 & COX-2). It also inhibits 5-lipoxygenase enzyme to suppress leukotriene (involved in inflammatory response) production.

### Cat's Claw

Uncaria tomentosa, also known as cat's claw, is an indigenous herb, Uncariae cortex consists of the dried stem bark, belonging to the family Rubiaceae, which had been used by South American natives for immunity problems as in infections, allergies, rheumatoid arthritis, lupus, and cancer.

It contains pentacyclic oxindole alkaloids. The medicinal parts are the root and the bark. The active ingredient is an alkaloid called rhynchophylline. Its beneficial effects on arthritis are due to both its anti-inflammatory and immuno-modulation actions. Cat's Claw can at various dosages on lymphocyte proliferation and nitric oxide expression in osteoarthritis and inhibit prostaglandin E2 and tumor necrosis factor alpha (TNF-α) which are mediators of the inflammatory process.

Devil's Claw

Devil's claw (Harpagophytum procumbent) is also known as Grapple plant, is a desert plant and is useful in pain and inflammation in arthritic conditions.

Devil's claw contains iridoid glycosides, primarily harpagoside which has demonstrated anti-inflammatory effects. Harpagoside inhibits both the cyclooxygenase (COX-2) and lipoxygenase inflammatory pathways.

EP Publication No. 0 552 657 A1 discloses that pure boswellic acid, physiologically acceptable salts thereof, derivatives thereof and salts of the derivatives or a boswellic acid-containing vegetable preparation may combat inflammatory processes caused by an increased leukotriene formation. The compounds used in particular for treating inflammatory arthropathies, epidermal lesions, allergic and chronic asthma, endotoxin shock, inflammatory bowel diseases and chronic hepatitis and also discloses the use of boswellic acid for treating the inflammatory processes alone or in combination with the other herbal medicines.

U.S. Pat. No. 5,827,528 A refers to medical adhesive composition comprising, as essential components, a pressure sensitive adhesive component and a water absorbing component, the pressure sensitive adhesive component consisting of an elastomer comprised of a thermoplastic elastomer and an elastomer having a low compatibility with the thermoplastic elastomer, and a softner containing at least a liquid rubber and a tackifier, wherein the thermoplastic elastomer of the pressure sensitive adhesive component is a styrene copolymer wherein the styrene copolymer two or more of SIS (styrene-isoprene-styrene block copolymer), SBS (styrene-butadiene-styrene block copolymer), SEBS (styrene-ethylene-butylene-styrene block copolymer), and SEPS (hydrogenated styrene-isoprene block copolymer) and thermoplastic elastomer is used as a base, into which mineral oil, a hydrocolloid component, plasticizer and a tackifier are added.

U.S. Pat. No. 5,888,514 A discloses a composition for treating a mammal having a condition characterized by bone or joint inflammation where extracts of Boswellia serrata, black currant seed oil and devil's claw powder are used in combination along with other inflammatory agents.

U.S. Pat. No. 6,096,334 A discloses a non-occlusive biomedical adhesive patch to be applied to the skin of a patient for therapeutic purposes comprising backing layer comprising a flexible sheet of water-insoluble material, flexible pressure-sensitive adhesive layer, pressure-sensitive adhesive, backing layer and removable liner.

U.S. Pat. No. 6,448,303 B1 discloses hot melt pressure sensitive adhesive especially suited for adhesive skin application, including transdermal drug delivery applications. The adhesive is used as a carrier contact adhesive or overlay contact adhesive for transdermal patches.

U.S. Pat. No. 6,579,543 B1 discloses the topical composition for application to an animal's skin for relief from a variety of symptoms caused by medical conditions or physical injuries wherein the composition comprises devil's claw root, evening primrose oil and ginger as analgesics, Boswellia, licorice as anti-inflammatory agent and other ingredients and combinations thereof.

U.S. Pat. No. 7,064,242 B2 discloses a patch comprising a backing layer and a adhesive layer formed on the back face of the backing layer wherein a woven fabric or a nonwoven fabric made of a rayon and pulp fiber mixture is employed as the backing layer and the mixing ratio thereof is from 3:7 to 7:3. It also discloses ginger extract, glycyrrhizinate as a plant extract.

U.S. Pat. No. 7,758,903 B2 discloses compositions which comprise at least one ingredient chosen from rosehips, blueberry, blackberry, elderberry, cranberry, rosemary, clove, feverfew, nettle root, artichoke, reishi mushroom, olive extract, green tea extract (epigallocatechin gallate), grape seed extract, resveratrol, viniferin, Aframomum melegueta, boswellia serrata extract, boswellia forte, ipriflavone, tocotrienols, evening primrose oil, INM-176, borage oil, krill oil, at least one type of xanthophyll (e.g., astaxanthin), green coffee extract (chlorogenic acid), and ferulic acid.

US publication No. 2013/0052271 A1 discloses the composition for the treatment of pain comprising Boswellia, ginger root, licorice, evening primrose oil, devil's claw root and Cat's Claw as anti-inflammatory agents and composition contains buffer, surfactant, thickening agent, vitamin, emulsifier, preservative, solvent, bulking agent, topical base composition, moisturizer, humectant gelling agent, and essential oil, d-alpha Tocopherol as anti-oxidant.

Inventi Impact NDDS, Vol. 2015, Issue 4, pp 202-212, discloses Boswellic Acid Transdermal Drug Delivery System for Arthritis comprising HPMC E6, oleic acid, HPMC E5, Ethylcellulose, PVP K30, PVA and Tween 20, PEG 400 and Glycerol, DMSO, Methanol and chloroform by Solvent Evaporation Technique.

PCT publication No. WO 2018/020512 A1 discloses the composition for slow/sustained/controlled release of at last one active ingredient wherein the active ingredients are selected from natural phytochemicals, phytochemical is selected from all hydrophobic and hydrophilic natural compounds, but not limited to, Lutein, Caffeine, Resveratrol, Berberin, 95% Curcuminoids, Gingerols, Bacosides, Boswellic

Acids, Chlorogenic Acids combinations thereof.

### Hot-Melt Coating Technology

In recent years, use of hot melt adhesives plays important role in transdermal drug delivery devices due to their superior physico-chemical properties. In solvent based technique mostly adhesives like PIB, acrylate, silicone derivatives are being used which controls the drug release by functional group interaction between drug and adhesive. Since herbal extracts contain high amount of oily substances, which tend to show cold flow property which in turn lead to less tack and poor adherence to skin when patches formulated using solvent-based coating technique. On the other hand, drugs that are not stable in aqueous matrix cannot be formulated by water based hydrogel technique.

In addition, Hot-melt coating technology has several advantages; firstly, HMC is economical and relatively faster process compared to solvent or water-based coatings. Secondly, HMC is particularly suitable for combined adhesive/drug-matrix device as they can be formulated to contain little or no chemical functional group. This reduces the possibility of medication/adhesive interactions and skin irritation. Thirdly, hot melt adhesive is less prone to swelling when in contact with alcohols used in certain medications, which is a particular problem with acrylics.

The boswellia extract is available in market in different combinations and different dosage forms like Rhuval oil which contains acetyl-keto-beta-boswellic acid in combination with wintergreen oil and bakuchiol. The polyherbal transdermal patch comprising combination of boswellia extract, evening primrose oil, blackcurrant seed oil, Ginger, Licorice, Cat's claw and Devil's claw is not available in the market.

All the prior art references related to the use of polyherbal extracts in preparing hydrophilic pressure sensitive hot melt adhesive and their use in treating or managing pain by proving synergistic effect in combination with anti-inflammatory herbs.

However, the inventors of present invention provide composition of transdermal patch using combination of polyherbal extracts including boswellia serrata extract, Evening primrose oil, Blackcurrant seed oil, Ginger, Licorice, Cat's claw and Devil's claw and pharmaceutically acceptable excipients. The inventors of present invention also provide an efficient process for the preparation of polyherbal transdermal patch of boswellia using hot-melt coating (HMC) technology comprising the steps of melting, mixing, coating, laminating, cutting, pouching and labelling.

### OBJECTIVE OF INVENTION

The main objective of the present invention is to provide a composition of polyherbal transdermal patch comprising boswellia extract, Evening primrose oil, Blackcurrant seed oil, Ginger, Licorice, Cat's claw and Devil's claw and permeation enhancer, hot melt adhesives, anti-oxidant, tackifier and vehicle and other pharmaceutically acceptable excipients.

Another objective of the present invention is to provide a composition of polyherbal transdermal patch comprising boswellia extract, Evening primroseoil, Blackcurrant seed oil, Ginger, Licorice, Cat's claw and Devil's claw, isopropyl myristate as permeation enhancers, (styrene isoprene thermoplastic elastomer)-SIS 5002 and pressen 1471 as hot melt adhesives, arkon-P 100 as tackifier mineral oil as vehicle and tocopheryl acetate as anti-oxidant and other pharmaceutically acceptable excipients.

Still another objective of the present invention is to provide a process for the preparation of polyherbal transdermal patch by using hot-melt coating technique (HMC) comprising the steps of melting, mixing, coating, laminating cutting, pouching and labelling.

Still another objective of the present invention is to provide a process for the preparation of transdermal patch comprising steps of melting hot-melt adhesives, adding tackifier to molten adhesive, then addition of all herbal actives along with mineral oil, isopropyl myristate and tocopheryl acetate by stirring to form hot molten base, coating, laminating and cutting into desired size.

Still another objective of the present invention is to provide an improved/efficient manufacturing process for preparation of transdermal patch by hot-melt coating technique, which is a solvent-free technique, faster and more economic coating process. In addition, HMC technique has several advantages including drug release, adhesiveness (tack) and physical properties of patch can be tuned relatively easy compared to solvent based coating technique.

In yet another objective of the present invention is to provide relief from pain by application of polyherbal transdermal patch.

### SUMMARY OF INVENTION

Accordingly, the present invention provides composition of polyherbal transdermal patch useful in providing relief from pain.

In one embodiment, the present invention is to provide a composition of polyherbal transdermal patch comprising combination of natural herbal extracts as active ingredients and pharmaceutically acceptable excipients in the treatment/management of pain.

In another embodiment, the present invention is to provide a composition of polyherbal transdermal patch comprising combination of natural herbal extracts which includes boswellia extract, evening primrose oil, blackcurrant seed oil, Ginger, Licorice, Cat's claw and Devil's claw and permeation enhancer, hot melt adhesives, tackifier, anti-oxidant and vehicle and other pharmaceutically acceptable excipients.

In another embodiment, the present invention is to provide a composition of polyherbal transdermal patch comprising boswellia extract, evening primrose oil, blackcurrant seed oil, ginger, licorice, cat's claw and devil's claw as active ingredients and permeation enhancer, hot melt adhesives, tackifier, vehicle, antioxidant and other pharmaceutically acceptable excipients for treatment/management of pain.

In another embodiment, the present invention provides a composition of polyherbal transdermal patch comprising boswellia extract, evening primrose oil, blackcurrant seed oil, ginger, licorice, cat's claw and devil's claw as active ingredients, isopropyl myristate as permeation enhancer, (styrene isoprene thermoplastic elastomer)-SIS 5002 and pressen 1471 as hot melt adhesives, Arkon-P 100 as tackifier, tocopheryl acetate anti-oxidant and mineral oil as vehicle and other pharmaceutically acceptable excipients.

In another embodiment, the present invention provides a process for the preparation of polyherbal transdermal patch by using hot-melt coating technique (HMC) comprising the steps of melting, mixing, coating, laminating and cutting.

In yet another embodiment, the present invention provides a polyherbal transdermal patch composition comprising:
0.1% to 15% (w/w) of two or more natural herbal extracts as active ingredients,
0.5% to 5% (w/w) of permeation enhancer,
0.1% to 3% of anti-oxidant,
15% to 90% (w/w) of hot melt adhesives,
1% to 10% (w/w) of tackifying material,
0.5% to 20% (w/w) of vehicle, and
0.1% to 10% (w/w) other pharmaceutically acceptable ingredients.

In yet another embodiment, the present invention provides a polyherbal transdermal patch composition comprising:
0.1% to 5% (w/w) of boswellia extract,
0.1% to 5% (w/w) evening primrose oil,
0.1% to 5% (w/w) blackcurrant seed oil,
0.1% to 5% (w/w) of licorice,
0.1% to 5% (w/w) of ginger,
0.1% to 5% (w/w) of cat's claw,
0.1% to 5% (w/w) of devil's claw,
0.5% to 5% (w/w) of isopropyl myristate,
0.1% to 3% of tocopheryl acetate,
15 to 90% (w/w) of of SIS 5002 and pressen 1471,
1 to 10% (w/w) of arkon-P 100,
0.5 to 20% (w/w) of mineral oil, and
0.1% to 10% (w/w) other pharmaceutically acceptable ingredients.

In still another embodiment, the present invention provides an improved/efficient manufacturing process for preparation of transdermal patch by hot-melt coating technique, which is a solvent-free technique, faster and more economic coating process. In addition, HMC technique has several advantages including drug release, adhesiveness (tack) and physical properties of patch can be tuned relatively easy compared to solvent based coating technique.

In yet another embodiment of the present invention provides a process for preparing polyherbal transdermal patch, the process comprising steps of:
a. melting hot melt adhesives under stirring at 100° C.-220° C.,
b. adding vehicle and tackifier to molten adhesive base under stirring at 100° C.-220° C.,
c. adding active ingredients along with permeation enhancer and anti-oxidant to molten adhesive blend under stirring to obtain homogenous material,
d. coating on to the polyethylene terephthalate release liner
e. laminating the obtained adhesive matrix,
f. cutting into desired size to get transdermal patch, pouching and labelling.

In yet another embodiment of the present invention provides a process for preparing polyherbal transdermal patch, the process comprising steps of:
a. melting SIS 5002 and Pressen 1471 under stirring at 100-220° C.,
b. adding mineral oil and arkon-P 100 to molten adhesive under stirring at 100-220° C.,
c. adding boswellia extract, evening primrose oil, blackcurrant seed oil, ginger, licorice, cat's claw and devil's claw along with isopropyl myristate and tocopheryl acetate to molten adhesive blend under stirring to obtain homogenous material,
d. coating on to the polyethylene terephthalate release liner,
e. laminating the obtained adhesive matrix, and
f. cutting into desired size to get transdermal patch, pouching and labelling.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The present invention provides composition of polyherbal transdermal patch comprising natural herbal extracts as active ingredients and pharmaceutically acceptable excipients.

The present invention provides composition of polyherbal transdermal patch comprising natural herbal extracts includes boswellia extract, evening primrose oil, blackcurrant seed oil, Ginger, Licorice, Cat's claw and Devil's claw and pharmaceutically acceptable excipients.

The present invention provides composition of polyherbal transdermal patch comprising boswellia extract, Evening primrose oil, Blackcurrant seed oil, Ginger, Licorice, Cat's claw and Devil's claw as natural herbal extracts, Isopropyl myristate as permeation enhancer, (styrene isoprene thermoplastic elastomer)-SIS 5002 and pressen 1471 as hot melt adhesives, arkon-P 100 as tackifier, Tocopheryl acetate as anti-oxidant, mineral oil as vehicle and other pharmaceutically acceptable excipients.

The term “active ingredients” of the present invention is used to relieve from pain conditions. Preferably used active ingredients are boswellia extract, Evening primroseoil, Blackcurrant seed oil, Ginger, Licorice, Cat's claw and Devil's claw.

The combination of Boswellic acid, Evening primrose oil, Blackcurrant seed oil, Ginger, Licorice, Cat's claw and Devil's claw are selected as they possess the analgesic and anti-inflammatory properties and proven to be safe, effective and also produces synergistic action.

The term “hot melt adhesives” of the present invention includes combination of one or more hot melt adhesives and includes at least two adhesives selected from the group of ethylene-vinyl acetate copolymer series (EVA hot melt adhesive), synthetic rubber-based hot melt adhesives, polyolefin based hot melt adhesive, polyamide based hot melt adhesive, polyester-based hot melt adhesives, polyurethane-based hot melt adhesives, styrene isoprene thermoplastic elastomer. Preferably, hot melt adhesives are (styrene isoprene thermoplastic elastomer)-SIS 5002 and pressen 1471.

Hot melt adhesives used in the polyherbal transdermal patch is in the range of 15 to 90% (w/w).

Permeation enhancer used in the composition of the present invention include, but are not limited to azones, isopropyl myristate, fatty acids, menthol, essential oils, terpenes, terpenoids, N-methyl-2-pyrrolidone, 1-dodecyl-azacycloheptan-2-one, oleic acid, oleyl alcohol, linoleic acid, isopropyl linoleate, butanediol, lauryl alcohol, lauryl acetate, lauryl lactate, ethyl acetate, dimethyl isosorbide, isostearic acid. Preferably, the permeation enhancer is isopropyl myristate.

Permeation enhancer used in the polyherbal transdermal patch composition is in the range of 0.5 to 5% (w/w), more preferably in the range of 0.5 to 3% of the total weight of the composition.

Tackifier used in the composition of the present invention include, but are not limited to petroleum resins (e.g., aliphatic hydrocarbon resins, alicyclic hydrocarbon resins, and aromatic hydrocarbon resins), phenolic resins, xylene resins and coumarone indene resins rosin derivatives (e.g., rosin, glycerin esters of rosin, hydrogenated rosins, glycerin esters of hydrogenated rosin, pentaerythritol esters of rosin, etc.), saturated alicyclic hydrocarbon resins (e.g., ARKON P-100), aliphatic hydrocarbon resins (e.g., Quintone B170) terpene resins (e.g., Clearon P-125), maleic acid resins and the like. Preferably, the tackifier is saturated alicyclic hydrocarbon resins arkon-P 100.

Tackifier used in the polyherbal transdermal patch composition is in the range of 1 to 10% (w/w) of the total weight of the composition, more preferably in the range of 1 to 5% (w/w) of the total weight of the composition.

Anti-oxidant used in the composition of the present invention includes, but not limited to Alpha Tocopherol, tocopheryl acetate, vitamin E derivatives, Ascorbic Acid, Ascorbyl Palmitate, vitamin C, Butylated Hydroxytoluene (BHT), Butylated Hydroxyanisole (BHA), Erythorbic Acid, Lanolin, Citric Acid Monohydrate. Preferably, the anti-oxidant is tocopheryl acetate.

Anti-oxidant used in the polyherbal transdermal patch composition is in the range of 0.1 to 3% (w/w) of the total weight of the composition, more preferably in the range of 0.1 to 0.6% (w/w) of the total weight of the composition.

“Vehicle” as used herein refer to carrier materials suitable for transdermal drug administration, and include any such materials known in the art, i.e., any liquid gel, solvent, liquid diluent, adhesive, or the like, which is nontoxic and which does not interact with other components of the composition in a deleterious manner. Vehicle, which also may function as solvents in some instances, are used to provide the compositions of the invention in their preferred form. Examples include, but not limited to, water, ethanol, propanol, isopropanol, mineral oil, silicone, polyethylene glycol, polypropylene glycol, liquid sugars, waxes, petroleum jelly and a variety of other oils, aloe and polymeric materials along with polyacrylate, silicone, natural and synthetic rubbers or other adhesives. Preferably, the vehicle is mineral oil.

Vehicle used in the polyherbal transdermal patch composition is in the range of 0.5 to 20% (w/w), more preferably in the range of 0.5 to 10% (w/w) of the total weight of the composition.

The polyherbal transdermal patch of the present invention has been prepared by hot melt coating technique. The advantage of this technique is simple and easy to manufacture, more economical and solvent free technique. Using HMC technique tuning drug release, adhesiveness (tack) and physical properties of patch is relatively good compared to solvent-based coating technique.

The polyherbal transdermal patch of the present invention has been prepared by hot melt coating technique using polyethylene terephthalate release liner and coated layer is laminated using nonwoven or woven fabric backing material.

The molten adhesive blend preparation comprising the SIS and pressen 1471 as hot melt adhesives. The physical & mechanical properties of adhesive matrix are achieved only with the combination of SIS 5002 and Pressen 1471 to get the desired adhesion & flexibility to adhesive matrix.

The content of SIS and pressen 1471 should contain 20% to 90% by mass with respect to total mass of transdermal patch. If the content falls within this range, the cohesive property and tack of adhesive layer can be maintained. Accordingly, favorable application properties can be obtained.

For the polyherbal transdermal patch preparation of present invention, it is preferred that the permeation enhancer used to enhance the permeation of active ingredients through the skin to provide better and fast therapeutic action. The concentration of permeation enhancer should be in the range of 0.1-3% by mass with respect to total mass of the adhesive matrix.

For the polyherbal transdermal patch preparation of present invention, it is preferred that the tackifier play a key role to maintain optimum sticking to skin. The physical and mechanical properties of transdermal patch are achieved only with the optimized concentration of tackifier to get the desired tackiness & flexibility. The concentration of tackifier should be in the range of 1-10% by mass with respect to total mass of adhesive matrix.

For the transdermal patch preparation of present invention, it is preferred that the vehicle should be in the range of 0.5-20%.

Various properties associated with each component of the transdermal patch compositions may affect the properties of the final product. Properties associated with the selection of raw materials, molecular weight, concentration and viscosity may influence the adhesive matrix formation, adhesion and therapeutic effect.

The invention disclosed herein is process for the preparation of transdermal patch useful for treatment/management of pain.

### Manufacturing Process for Polyherbal Transdermal Patch:

1. Preparation of Hot Melt Adhesive Blend

The hot melt adhesive blend is prepared by melting of Pressen 1471 and SIS under stirring preferably at 100° C.-220° C. temperature. Later, to the molten adhesive mineral oil and Arkon were added under stirring to obtain homogenous adhesive blend. Preferably, the concentration of pressen 1471 should be in the range of 20-80% (w/w), preferably, SIS should be in the range of 5-30% (w/w), preferably, mineral oil should be in the range of 0.5-20% (w/w), preferably, Arkon should be in the range of 1-10% (w/w).

2. Addition of Active Ingredients

Boswellia extract, evening primrose oil, blackcurrant seed oil, ginger, licorice, cat's claw and devil's claw were added along with isopropyl myristate and tocopheryl acetate to the molten adhesive blend. Preferably, the concentration of boswellia extract should be in the range of 0.1-5% (w/w), preferably, the concentration of evening primrose oil should be in the range of 0.1-5% (w/w), preferably, the concentration of black currant seed oil should be in the range of 0.1-5% (w/w), preferably, the concentration of licorice should be in the range of 0.1-5% (w/w), preferably, the concentration of ginger should be in the range of 0.1-5% (w/w), preferably, the concentration of cat's claw should be in the range of 0.1-5% (w/w), preferably, the concentration of devil's claw should be in the range of 0.1-5% (w/w), preferably, the concentration of isopropyl myristate should be in the range of 0.5-5% (w/w), preferably, the concentration of tocopheryl acetate should be in the range of 0.1-3% (w/w).

3. Coating

The hot melt adhesive blend was uniformly coated with desired thickness on to the polyethylene terephthalate release liner.

4. Lamination and Cutting and Packaging

Then coated adhesive matrix was laminated using Nonwoven fabric.

5. Cutting and Packaging

Then the resulting product cut into a desired size to produce transdermal patch and finally packed in triple laminated aluminum pouch.

Formulations were developed using different concentrations of pressen 1471, SIS, and Arkon. The formulations prepared with different variations were evaluated for their description, adhesion (tack), peel test, assay.

The following examples describes the nature of the invention and are given only for the purpose of illustrating the present invention in more detail and are not limitative and relate to solutions, which have been particularly effective on bench scale.

### EXAMPLE 1

| | | Concen- | |
|---|---|---|---|
| S. | | tration | mg/ |
| No. | Ingredients | (%) | patch |
| 1. | Sallaki (Boswellia Serrata) | 0.706 | 12 |
| 2. | Evening primrose oil (Oenothera biennis) | 1.471 | 25 |
| 3. | Blackcurrant seed oil (Ribes nigrum) | 1.471 | 25 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3.000 | 51 |
| 9. | (Styrene Isoprene styrene thermoplastic | 19.118 | 325 |
| | elastomer) - SIS 5002 | | |
| 10. | Pressen 1471 | 65.882 | 1120 |
| 11. | Arkon-P 100 | 4.529 | 77 |
| 12. | Mineral oil | 0.882 | 15 |
| 13. | Tocopheryl acetate | 0.529 | 9 |

### EXAMPLE 2

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 1.471 | 25 |
| 3. | Blackcurrant seed oil (Ribes nigrum) | 1.471 | 25 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3.000 | 51 |
| 9. | (Styrene Isoprene styrene thermoplastic | 17.647 | 300 |
| | elastomer) - SIS 5002 | | |
| 10. | Pressen 1471 | 66.941 | 1138 |
| 11. | Arkon-P 100 | 4.529 | 77 |
| 12. | Mineral oil | 0.529 | 9 |
| 13. | Tocopheryl acetate | 0.529 | 9 |

### EXAMPLE 3

| | | Concen- | |
|---|---|---|---|
| S. | | tration | mg/ |
| No. | Ingredients | (%) | patch |
| 1. | Sallaki (Boswellia Serrata) | 2.206 | 37.5 |
| 2. | Evening primrose oil (Oenothera biennis) | 1.471 | 25 |
| 3. | Blackcurrant seed oil (Ribes nigrum) | 1.471 | 25 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3 | 51 |
| 9. | (Styrene Isoprene styrene thermoplastic | 19.147 | 325.5 |
| | elastomer) - SIS 5002 | | |
| 10. | Pressen 1471 | 64.706 | 1100 |
| 11. | Arkon-P 100 | 4.529 | 77 |
| 12. | Mineral oil | 0.529 | 9 |
| 13. | Tocopheryl acetate | 0.529 | 9 |

### EXAMPLE 4

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 0.147 | 2.5 |
| 3. | Blackcurrant seed oil (Ribes nigrum) | 2.794 | 47.5 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3.000 | 51 |
| 9. | (Styrene Isoprene styrene thermoplastic | 17.765 | 302 |
| | elastomer) - SIS 5002 | | |
| 10. | Pressen 1471 | 66.471 | 1130 |
| 11. | Arkon-P 100 | 4.529 | 77 |
| 12. | Mineral oil | 0.882 | 15 |
| 13. | Tocopheryl acetate | 0.529 | 9 |

### EXAMPLE 5

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 2.794 | 47.5 |
| 3. | Blackcurrant seed oil (Ribes nigrum) | 0.147 | 2.5 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3.000 | 51 |
| 9. | (Styrene Isoprene styrene thermoplastic | 17.882 | 304 |
| | elastomer) - SIS 5002 | | |
| 10. | Pressen 1471 | 66.706 | 1134 |
| 11. | Arkon-P 100 | 4.529 | 77 |
| 12. | Mineral oil | 0.529 | 9 |
| 13. | Tocopheryl acetate | 0.529 | 9 |

### EXAMPLE 6

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 1.471 | 25 |
| 3. | Blackcurrant seed oil (Ribes nigrum) | 1.471 | 25 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3 | 51 |
| 9. | (Styrene Isoprene styrene thermoplastic | 18.235 | 310 |
| | elastomer) - SIS 5002 | | |
| 10. | Pressen 1471 | 66.353 | 1128 |
| 11. | Arkon-P 100 | 4.529 | 77 |
| 12. | Mineral oil | 0.529 | 9 |
| 13. | Tocopheryl acetate | 0.529 | 9 |

### EXAMPLE 7

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 0.735 | 12.5 |
| 3. | Blackcurrant seed oil (Ribes nigrum) | 2.206 | 37.5 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3 | 51 |
| 9. | (Styrene Isoprene styrene thermoplastic | 19.147 | 325.5 |
| | elastomer) - SIS 5002 | | |
| 10. | Pressen 1471 | 65.441 | 1112.5 |
| 11. | Arkon-P 100 | 4.529 | 77 |
| 12. | Mineral oil | 0.529 | 9 |
| 13. | Tocopheryl acetate | 0.529 | 9 |

### EXAMPLE 8

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 2.206 | 37.5 |
| 3. | Blackcurrant seed oil (Ribes nigrum) | 0.735 | 12.5 |
| 4. | licorice (Glycyrrhiza glabra) | 0.412 | 7 |
| 5. | Ginger (Zingiber officinale) | 1.471 | 25 |
| 6. | Cat's claw (Uncaria tomentosa) | 0.176 | 3 |
| 7. | Devil's claw (Harpagophytum procumbens) | 0.353 | 6 |
| 8. | Isopropyl myristate | 3 | 51 |
| 9. | (Styrene Isoprene styrene thermoplastic | 20.588 | 350 |
| | elastomer) - SIS 5002 | | |
| 10. | Pressen 1471 | 64.000 | 1088 |
| 11. | Arkon-P 100 | 4.529 | 77 |
| 12. | Mineral oil | 0.529 | 9 |
| 13. | Tocopheryl acetate | 0.529 | 9 |

### EXAMPLE 9

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 1.471 | 25 |
| 3. | Blackcurrant seed oil (Ribes nigrum) | 1.471 | 25 |
| 4. | licorice (Glycyrrhiza glabra) | 0.206 | 3.5 |
| 5. | Ginger (Zingiber officinale) | 0.735 | 12.5 |
| 6. | Cat's claw (Uncaria tomentosa) | 0.088 | 1.5 |
| 7. | Devil's claw (Harpagophytum procumbens) | 0.235 | 4 |
| 8. | Isopropyl myristate | 3 | 51 |
| 9. | (Styrene Isoprene styrene thermoplastic | 18.676 | 317.5 |
| | elastomer) - SIS 5002 | | |
| 10. | Pressen 1471 | 67.059 | 1140 |
| 11. | Arkon-P 100 | 4.529 | 77 |
| 12. | Mineral oil | 0.529 | 9 |
| 13. | Tocopheryl acetate | 0.529 | 9 |

### EXAMPLE 10

| | | Concen- | |
|---|---|---|---|
| S. | | tration | mg/ |
| No. | Ingredients | (%) | patch |
| 1. | Sallaki (Boswellia Serrata) | 1.471 | 25 |
| 2. | Evening primrose oil (Oenothera biennis) | 1.471 | 25 |
| 3. | Blackcurrant seed oil (Ribes nigrum) | 1.471 | 25 |
| 4. | licorice (Glycyrrhiza glabra) | 0.588 | 10 |
| 5. | Ginger (Zingiber officinale) | 2.206 | 37.5 |
| 6. | Cat's claw (Uncaria tomentosa) | 0.265 | 4.5 |
| 7. | Devil's claw (Harpagophytum procumbens) | 0.471 | 8 |
| 8. | Isopropyl myristate | 3 | 51 |
| 9. | (Styrene Isoprene styrene thermoplastic | 17.941 | 305 |
| | elastomer) - SIS 5002 | | |
| 10. | Pressen 1471 | 65.529 | 1114 |
| 11. | Arkon-P 100 | 4.529 | 77 |
| 12. | Mineral oil | 0.529 | 9 |
| 13. | Tocopheryl acetate | 0.529 | 9 |

### Manufacturing Process for Polyherbal Transdermal Patch:

Required quantity of pressen 1471 and SIS, were molten in hot vessel at 100° C.-220° C. under stirring.

Required quantity of arkon and mineral oil were added to molten adhesive blend under stirring at 100° C.-220° C. temperature to obtain homogenous blend.

Required quantity of all herbal extracts, boswellia, evening primrose oil, blackcurrant seed oil, licorice, ginger, cat's claw and devil's claw along with isopropyl myristate and tocopheryl acetate were added to above molten adhesive base under stirring to obtain homogenous mixture of active ingredients in blend for coating.

Polyherbal transdermal patch prepared as per the Example no. 5 of the present invention has shown good stability among all of the examples given of the present invention under different stability conditions. The Polyherbal transdermal patch was evaluated at different stability conditions i.e., at 40° C./75% RH, 30° C./75% RH and 25° C./60% RH, The observed for physical properties of patch, examined its total content of boswellic acids by assay, uniformity of Dosage Units (by Content Uniformity), Tack Test, Peel Test, Microbial Enumeration Limit and Total Aerobic Microbial Count (cfu/gm) including Total yeasts and molds count (cfu/gm), Pseudomonas aeruginosa (gm) and Staphylococcus aureus (gm) ans data is given below tables 1, 2 & 3:

TABLE 1
Stability Condition: 40° C./75% RH (Example 5)

| Test | Specification | Initial | 3 Month | 6 Month |
|---|---|---|---|---|
| Description | Brown to dark brown | Complies | Complies | Complies |
| | coloured matrix type | | | |
| | transdermal patch that is | | | |
| | rectangular shape patch | | | |
| Assay | Each patch contains 25 | 100.02 | 97.7 | 98.5 |
| Total boswellic acids | mg of boswellic acids | | | |
| | Limit: 22.5 mg to 27.5 mg | | | |
| | i.e 90% to 110% of label claim | | | |
| Uniformity of Dosage Units | L1 ≤15 | 3.1 | 3.8 | 4.2 |
| (by Content Uniformity) | | | | |
| Tack Test | NLT 2.0 N | 4.1 | 3.8 | 3.6 |
| Peel Test | NLT 6.0 N | 9.7 | 9.2 | 9.5 |

Microbial Enumeration Limit:

| | | | | |
|---|---|---|---|---|
| Total Aerobic Microbial Count | Not more than 100 | Absent | NA | Absent |
| (cfu/gm) | | | | |
| Total yeasts and molds count | Not more than 10 | Absent | | Absent |
| (cfu/gm) | | | | |
| Pseudomonas aeruginosa (gm) | Absent | Absent | | Absent |
| Staphylococcus aureus (gm) | Absent | Absent | | Absent |

TABLE 2
Stability Condition: 30° C./75% RH (Example 5)

| Test | Specification | Initial | 3 Month | 6 Month |
|---|---|---|---|---|
| Description | Brown to dark brown | Complies | Complies | Complies |
| | coloured matrix type | | | |
| | transdermal patch that is | | | |
| | rectangular shape patch | | | |
| Assay | Each patch contains 25 | 100.02 | 98.3 | 99.2 |
| Total boswellic acids | mg of boswellic acids | | | |
| | Limit: 22.5 mg to 27.5 mg | | | |
| | i.e 90% to 110% of label claim | | | |
| Uniformity of Dosage Units | L1 ≤15 | 3.1 | 3.6 | 3.8 |
| (by Content Uniformity) | | | | |
| Tack Test | NLT 2.0 N | 4.1 | 3.1 | 3.8 |
| Peel Test | NLT 6.0 N | 9.7 | 9.1 | 8.2 |

Microbial Enumeration Limit

| | | | | |
|---|---|---|---|---|
| Total Aerobic Microbial Count | Not more than 100 | Absent | NA | Absent |
| (cfu/gm) | | | | |
| Total yeasts and molds count | Not more than 10 | Absent | | Absent |
| (cfu/gm) | | | | |
| Pseudomonas aeruginosa (gm) | Absent | Absent | | Absent |
| Staphylococcus aureus (gm) | Absent | Absent | | Absent |

TABLE 3
Stability Condition: 25° C./60% RH (Example 5)

| Test | Specification | Initial | 3 Month | 6 Month |
|---|---|---|---|---|
| Description | Brown to dark brown | Complies | Complies | Complies |
| | coloured matrix type | | | |
| | transdermal patch that is | | | |
| | rectangular shape patch | | | |
| Assay | Each patch contains 25 | 100.2 | 98.7 | 99.3 |
| Total boswellic acids | mg of boswellic acids | | | |
| | Limit: 22.5 mg to 27.5 mg | | | |
| | i.e 90% to 110% of label claim | | | |
| Uniformity of Dosage Units | L1 ≤15 | 3.1 | 3.2 | 3.5 |
| (by Content Uniformity) | | | | |
| Tack Test | NLT 2.0 N | 4.1 | 3.7 | 3.9 |
| Peel Test | NLT 6.0 N | 9.7 | 8.5 | 8.9 |

Microbial Enumeration Limit

| | | | | |
|---|---|---|---|---|
| Total Aerobic Microbial Count | Not more than 100 | Absent | NA | Absent |
| (cfu/gm) | | | | |
| Total yeasts and molds count | Not more than 10 | Absent | | Absent |
| (cfu/gm) | | | | |
| Pseudomonas aeruginosa (gm) | Absent | Absent | | Absent |
| Staphylococcus aureus (gm) | Absent | Absent | | Absent |

We claim:

1. A polyherbal transdermal patch composition comprising combination of natural herbal extracts includes boswellia extract, evening primrose oil, blackcurrant seed oil, Ginger, Licorice, Cat's claw and Devil's claw and permeation enhancer, hot melt adhesives, tackifier, anti-oxidant and vehicle and other pharmaceutically acceptable excipients.

2. The polyherbal transdermal patch composition as claimed in claim 1, wherein the composition comprising permeation enhancer which is selected from azones, isopropyl myristate, fatty acids, menthol, essential oils, terpenes, terpenoids, N-methyl-2-pyrrolidone, 1-dodecyl-azacycloheptan-2-one, oleic acid, oleyl alcohol, linoleic acid, isopropyl linoleate, butanediol, lauryl alcohol, lauryl acetate, lauryl lactate, ethyl acetate, dimethyl isosorbide, isostearic acid.

3. The polyherbal transdermal patch composition as claimed in claim 1, wherein the composition comprising hot melt adhesives is combination of one or more hot melt adhesives and includes at least two adhesives selected from the group of ethylene-vinyl acetate copolymer series (EVA hot melt adhesive), synthetic rubber-based hot melt adhesives, polyolefin based hot melt adhesive, polyamide based hot melt adhesive, polyester-based hot melt adhesives, polyurethane-based hot melt adhesives, styrene isoprene thermoplastic elastomer.

4. The polyherbal transdermal patch composition as claimed in claim 1, wherein the composition comprising tackifier is selected from petroleum resins (e.g., aliphatic hydrocarbon resins, alicyclic hydrocarbon resins, and aromatic hydrocarbon resins), phenolic resins, xylene resins and coumarone indene resins rosin derivatives (e.g., rosin, glycerin esters of rosin, hydrogenated rosins, glycerin esters of hydrogenated rosin, pentaerythritol esters of rosin, etc.), saturated alicyclic hydrocarbon resins, aliphatic hydrocarbon resins, terpene resins, maleic acid resins and the like.

5. The polyherbal transdermal patch composition as claimed in claim 1, wherein the composition comprising anti-oxidant is selected from Alpha Tocopherol, tocopheryl acetate, vitamin E derivatives, Ascorbic acid, Ascorbyl Palmitate, vitamin C, Butylated Hydroxytoluene (BHT), Butylated Hydroxyanisole (BHA), Erythorbic acid, Lanolin, Citric acid monohydrate.

6. The polyherbal transdermal patch composition as claimed in claim 1, wherein the composition comprising vehicle is selected from water, ethanol, propanol, isopropanol, mineral oil, silicone, polyethylene glycol, polypropylene glycol, liquid sugars, waxes, petroleum jelly, aloe and polymeric materials along with polyacrylate, silicone, natural and synthetic rubbers or other adhesives.

7. The polyherbal transdermal patch composition as claimed in claim 1, wherein the composition comprising:
0.1% to 15% (w/w) of two or more natural herbal extracts as active ingredients,
0.5% to 5% (w/w) of permeation enhancer,
0.1% to 3% of anti-oxidant,
15% to 90% (w/w) of hot melt adhesives,
1% to 10% (w/w) of tackifying material,
0.5% to 20% (w/w) of vehicle, and
0.1% to 10% (w/w) other pharmaceutically acceptable ingredients.

8. The polyherbal transdermal patch composition as claimed in claim 7, wherein the composition comprising boswellia extract, evening primrose oil, blackcurrant seed oil, ginger, licorice, cat's claw and devil's claw are natural herbal extracts as active ingredients, isopropyl myristate as permeation enhancer, (styrene isoprene thermoplastic elastomer)-SIS 5002 and pressen 1471 as hot melt adhesives, Arkon-P 100 as tackifier, tocopheryl acetate as anti-oxidant and mineral oil as vehicle and other pharmaceutically acceptable excipients.

9. An improved/efficient manufacturing process for the preparation of polyherbal transdermal patch by using hot-melt coating technique (HMC) comprising the steps of melting, mixing, coating, laminating and cutting.

10. The process for preparing polyherbal transdermal patch as claimed in claim 9, wherein the process comprising steps of:
a. melting hot melt adhesives under stirring at 100° C.-220° C.,
b. adding vehicle and tackifier to molten adhesive base under stirring at 100° C.-220° C.,
c. adding active ingredients along with permeation enhancer and anti-oxidant to molten adhesive blend under stirring to obtain homogenous material,
d. coating on to the polyethylene terephthalate release liner
e. laminating the obtained adhesive matrix,
f. cutting into desired size to get transdermal patch, pouching and labelling.
